(19) United States
(12) Patent Application Publication
(10) Pub. No.: US 2023/0166101 A1
(43) Pub. Date: Jun. 1, 2023

(54) ELECTROCEUTICAL DRESSING FOR WOUND CARE

(71) Applicant: Ohio State Innovation Foundation, Columbus, OH (US)

(72) Inventors: Shaurya PRAKASH, Columbus, OH (US); Vishwanath SUBRAMANIAM, Westerville, OH (US); Traci WILGUS, Dublin, OH (US); Paul STOODLEY, Columbus, OH (US); Daniel WOZNIAK, Columbus, OH (US); Prashanth Mohana SUNDARAM, Columbus, OH (US); Rachel HEALD, Laurel, MD (US); Gurleen VILKHU, Lewis Center, OH (US); Kavya NARAYANAN, Dublin, OH (US)

(21) Appl. No.: 17/918,051

(22) PCT Filed: Apr. 8, 2021
(86) PCT No.: PCT/US2021/026414

§ 371 Date: Oct. 10, 2022

Related U.S. Application Data
(60) Provisional application No. 63/007,010, filed on Apr. 8, 2020.

(51) Int. Cl. A61N 1/04

(52) U.S. Cl. CPC A61N 1/0468

(57) ABSTRACT

Electroceutical dressings having at least three electrodes that are used for prevention and mitigation of biofilm and bacterial infection by an applied electric current are provided. Methods of making the dressings and methods of applying an electric current to promote the wound healing process are also disclosed.

### CROSS-REFERENCE TO RELATED APPLICATION

This application claims priority to and benefit of U.S. Provisional Pat. Application No. 63/007,010 filed Apr. 8, 2020, which is fully incorporated by reference and made a part hereof.

### TECHNICAL FIELD

The present disclosure is generally directed to devices and methods for generating and directing an electric current through a wound to promote healing. More specifically, the present disclosure is directed to devices that include dressings for applying an electric current through a wound, which can provide antimicrobial and antibiofilm effects and facilitate wound healing.

### BACKGROUND

There are two kinds of bacterial strains, (i) free-floating or planktonic and (ii) attached or sessile bacteria. Surface attachment provides additional protection for the bacteria, improves cell-cell interactions (quorum sensing), and help concentrate nutrients. A biofilm is a form of sessile bacteria, consisting of a dense colony of bacteria attached to a surface. A bacterial biofilm is defined as “a structured community of bacterial cells enclosed in a self-produced polymeric matrix and adherent to an inert or living surface,” (Costerton, J. W., Stewart, P. S., & Greenberg, E. P. (1999). Bacterial Biofilms: A Common Cause of Persistent Infections. Science, 284(5418), 1318-1322. doi: 10.1126/science.284.5418.1318). The polymeric matrix is connected with strong chemical bonds, resistant and highly adaptable to biocides, antibiotics, and physical stress. Examples of physical stress and other environmental conditions include extreme temperatures, pH changes, and exposure to ultraviolet light.

Common biofilm-forming bacteria include Pseudomonas aeruginosa and Staphylococcus epidermidis, both of which are commonly present in water, air, soil, and skin. According to the Center for Biofilm Engineering at Montana State University, biofilm forms when bacteria adhere to surfaces in moist environments by excreting a slimy, glue-like substance. This slimy excretion is referred to as the extracellular polymeric substance (EPS) which holds the bacteria in the biofilm matrix. The bacteria form a biofilm in three phases: attachment, growth, and dispersal.

A biofilm is a serious form of a bacterial infection because surface attachment and colonization provides additional protection against environmental changes, including antibiotic medications. The antibiotics in use today were created using studies of bacteria suspended in agar, or free-floating bacteria. However, it has been discovered in recent years that several bacteria preferentially attach to various substrates, both living and inert, and are highly adaptable organisms that exhibit survival skills in this form. Further, microbial biofilms are tolerant of antibiotic doses up to 1,000 times greater than those of planktonic bacteria (CBE).

Wound infections are not only expensive complications following surgery but still after many years are a major source of bacteria that drive the nosocomial infection rates in hospitals. These infections can complicate illness, cause anxiety, increase patient discomfort and can lead to death. In the biofilm form, bacteria can become recalcitrant to antimicrobials and host defenses, posing a rapidly escalating threat to human health. Typical antimicrobial and antibiotic treatments for these biofilm based infections run the risk of developing antimicrobial and antibiotic resistant strains of bacteria. There remains a need for biophysical treatments not subject to bacteria resistance.

Wound healing is a complex process involving a series of biochemical events, ranging from influx of macrophages and neutrophils to remodeling of the extracellular matrix (ECM). A range of studies have been carried out in past, showing the use of mechanical and electrical stimulations to hasten the wound healing process. It has been found that electrical stimulation has the ability to induce cellular migration and proliferation that may have important implications on wound healing. In addition to cellular changes, electrical stimulation has also been shown to kill bacteria, both planktonic and in biofilm form known to colonize on wounds and inhibit the healing process. It has been shown that by applying electrical stimulation using an electroceutical dress, infection can be mitigated by generating antimicrobials like hypochlorous acid (HOC1) and chloramines. Past work has also shown that electrical stimulation can play a major role in the directional migration of immune cells like lymphocytes, neutrophils, monocytes/macrophages. Moreover, HOC1 is also known to play an important role in the infiltration of activated leukocytes to the sites of inflammation. All these previous results suggest that by having a better control over the inflammatory response of cells, the wound healing processes could be influenced to orchestrate and/or potentially control specific steps in the complex cascade that is a healing wound. A healed wound is one where the open dermal area is closed and the barrier function of the epithelial layer is restored.

What is desired is a wound healing approach using an electroceutical dressing that provides periodic wound bed surveillance and, based on the state of the wound, control of the electroceutical actuation to orchestrate active interventions for faster healing rates by systematically controlling inflammation and vascularization. Specifically, what is desired is an electroceutical dressing that provides an integrated platform with multiple sensing modalities to actively orchestrate selective cell responses

### SUMMARY

Disclosed and described herein are electroceutical dressings for a wound. Specifically, described herein are embodiments of an electroceutical dressing that provide (1) better control on the electrochemistry by integrating a 3rd electrode on the substrate, (2) options to add one or more sensors enabling real-time tracking of wound healing and infection mitigation through wireless monitoring, and (3) a multi-layer design that can allow integration of additional components to translate a simple dressing to an integrated engineering platform or system.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 is a simplified electrical schematic of an electroceutical dressing for a wound;

FIG. 2A is a cross-section/profile view of an electroceutical dressing;

FIG. 2B is a plan view showing the wound side of an electroceutical dressing;

FIG. 2C is an illustration of an electroceutical dressing showing an on-board reservoir with appropriate tubing that is used to apply moisture to the wound while the electroceutical dressing is in place;

FIG. 2D is an illustration of an electroceutical dressing with all or a portion of the energy source separate from the substrate;

FIG. 2E illustrates another embodiment of an electroceutical dressing having an alternate form of an energy source;

FIG. 2F illustrates an embodiment of an electroceutical dressing such as the one shown in FIG. 2E, that further comprises a plurality of unconnected electrodes in a spaced pattern on the substrate that are electrically isolated from one another on the substrate;

FIG. 2G is an illustration of an electroceutical dressing that optionally includes a barrier that covers a part of or substantially covers a side of the substrate opposite the wound;

FIG. 2H illustrates an example of an electroceutical dressing that further comprise a current-limiting element;

FIG. 2I illustrates electrical impedance spectroscopy (EIS) measurements on exemplary pig burn wounds that can be used to determine healing in the wound;

FIG. 2J illustrates non-contact eddy current measurements were also performed on exemplary pig burn wounds, which can also be used to determine wound healing;

FIG. 2K is an illustration that shows biofilm formed in the wound;

FIGS. 3A-3G are non-limiting examples of various size, spacing and shapes of conductive anodes;

FIGS. 4A and 4B illustrate that sensors (e.g., eddy current, micro-electrochemical impedance, etc.) can be used to track wound closure through electrical property changes in burn wounds as they heal is a SolidWorks model of an exemplary bandage and battery system;

FIG. 5A is a photograph of an embodiment of the electroceutical dressing interfaced with Bluetooth transmission circuit and the electronic device (in this case mobile) receiving the data from the Bluetooth;

FIG. 5B illustrates current data recorded by the Bluetooth and compared against the current data recorded by a picoammeter; and

FIG. 6 is an illustration of an exemplary three-electrode electroceutical dressing.

### DETAILED DESCRIPTION

In the United States, 6.5 million patients are affected by chronic wounds, sometimes complicated by infection. If the bacteria form a biofilm at the wound site, treatment of the infection becomes significantly more difficult. Biofilm bacteria are 500 to 5,000 times more resistant to antibiotic medications than the non-biofilm bacteria. Previous studies have shown that the presence of direct electric current through the biofilm enhances the activity of various antibiotics against biofilm-forming bacterial strains such as Pseudomonas aeruginosa and Staphylococcus epidermidis. This behavior has been referred to as the electro-bactericidal effect.

Disclosed herein are electroceutical wound care dressings and methods of using the electroceutical wound care dressings for wound treatment in humans and animals. The electroceutical wound care dressings can be used to apply an electric current through a wound to aid in bacterial infection prevention and destruction. These dressings can provide a functional antimicrobial and antibiofilm barrier.

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes¬ from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the Examples included therein and to the Figures and their previous and following description.

In one aspect, disclosed are wound care dressings that include an electrode assembly and an electric current generating and control assembly. The electrode assembly can be used for applying an electric current for wound healing. The electric current generating and control assembly can be used to control and vary the electric current intensity during the wound healing period. The wound care dressings can provide antimicrobial and antibiofilm effects, which aid wound healing and tissue regeneration.

FIG. 1 is a simplified electrical schematic of an electroceutical dressing 100 for a wound. In this schematic, the wound 102 is represented as an electrical resistor. The dressing 100 comprises a substrate 104 and a plurality of electrodes positioned on or within the substrate 104. The substrate has two sides, a wound-facing side and a second side opposite the wound-facing side. The plurality of electrodes comprise at least a conductive anode 106 (also known as a “working electrode” in electrochemistry applications having more than two electrodes), a conductive cathode 108 (also called a “counter electrode”), and a conductive reference electrode 112. The reference electrode 112 follows rules of basic electrochemistry in being of a material that presents a known potential or allows measurement of potential drop between the working and reference electrode 112 with no current flow through the reference electrode 112. The flow of current may be limited by connecting to an external high impedance input. Each of three or more electrodes are configured to be in at least partial contact with a wound 102 when the electroceutical dressing is placed on the wound 102. Further comprising the schematic of an electroceutical dressing 100 is an energy source 110. In some instances, the energy source 110 comprises a flexible fabric energy source such as those described in U.S. Pat. Application Publication No. 2019/0247234 published Aug. 15, 2019, which is fully incorporated by reference and made a part hereof. The energy source 110 is generally connected to the electrode pair 106, 108. For example, the energy source 110 has a positive terminal and a negative terminal, wherein the positive terminal is configured to connect to one of the conductive anode 106 and/or the conductive cathode 108, and the negative terminal of the energy source 110 is configured to connect to an other of the conductive cathode 108 and/or the conductive anode 106 when the electroceutical dressing 100 is placed on the wound 102, wherein when connected to the conductive anode 106 and the conductive cathode 108, the energy source induces an electrical current to flow through the wound.

The anode 106, the cathode 108 and the conductive reference electrode 112 are electrically insulated from one another. The energy source 110 induces a voltage differential between the conductive anode 106 and conductive cathode 108, which causes an electrical current (I) to flow from the conductive anode 106, through the wound 102 (represented here as an electrical resistor), to the conductive cathode 108.

In some instances the reference electrode 112 is connected to a source with a known potential. For example, the reference electrode 112 may be connected to an electrical ground. The potential between the anode 106 and/or the cathode 108 and the reference electrode 112 can be determined when the reference electrode 112 is connected to the source having known potential. In this way, a potential difference between the conductive anode 106 and/or the conductive cathode 108 and the conductive reference node can be measured and the energy source can be used to maintain that potential difference at or above a desired potential to generate hypochlorous acid in the wound. For example, the desired potential difference may be 1.14 volts with respect to a Ag/AgCl reference electrode, or greater, which is a potential difference that is known to produce HOC1 electrochemically.

FIGS. 2A and 2B are physical representations of the schematic of an electroceutical dressing 100 for a wound. FIG. 2A is a cross-section/profile view of the electroceutical dressing 100 and FIG. 2B is a plan view showing the wound side of the electroceutical dressing 100. In FIG. 2B the energy source 110 and wiring is shown in dashed lines to represent that it is located on the substrate 104 on the side opposite the wound side. It is also to be appreciated that the energy source 110 may be located off of or remote from the substrate 104. Further, though FIGS. 2A and 2B illustrate a single electrode pair - anode 106 and cathode 108, and reference node 112; however, it is to be appreciated that the electroceutical dressing 100 may be comprised of any number of electrode pairs 106, 108 in electrical communication with energy source 110 or even a plurality of energy sources 110 that are equal to or less than the number of electrode pairs. In non-limiting examples, the electrodes 106, 108, 112 may have a thickness of 25 µm to 500 µm, 50 µm to 400 µm, 75 µm to 300 µm, or 100 µm to 200 µm and each of the anode 106, cathode and reference node may have a thickness that differs from at least one of the other anode 106, cathode 108 and reference node 112.

Also, though FIGS. 2A and 2B depict a singular, monolithic substrate 104, it is to be appreciated that the substrate 104 may be comprised of one, two or even more separate portions. For example, the electrodes 106, 108, 112 may be positioned on a singular substrate 104, or the substrate 104 may comprise a plurality of substrates and any one or more of the conductive anode 106, conductive cathode 108 and reference node 112 may be positioned on a first substrate while at least an other of the conductive anode 106, cathode 108, and reference node 12 may be positioned on a second substrate.

Generally, in regard to the substrate 104, it is comprised of material that is substantially electrically insulating. For example, the substrate 104 may be comprised of silk, polyester, and any material that has ability for printing a desired geometry and is compatible with a wound environment (i.e., does not occlude the wound for transport of essential fluids including oxygen) including polymeric substrates common to the medical industry like Polydimethylsiloxane (PDMS) and the like. In one embodiment of the dressings 100, the substrate 104 is comprised of silk and the silk comprises Habotai silk. In other embodiments the substrate 104 may be comprised of semiconductive materials or may have conductive elements within the substrate. For example, at least one of the conductive anode 106, the conductive cathode 108, or the reference node 112 may be woven into the substrate 104. In one specific example, at least one of the conductive anode 106, the conductive cathode 108, or the reference node 112 comprise a conductive silver material woven into a Habotai silk substrate 104. In one non-limiting example, an electrical current may be circulated through the conductive or semiconductive element of the substrate 104 in parallel to the current that flows from the conductive anode 106, through the wound 102, to the conductive cathode 108. The current through the substrate may create an electrical field that can facilitate healing of the wound. Generally, the substrate 104 or at least the wound side portion of the substrate 104 is sterile. Non-limiting examples of substrate 104 thickness include 10 µm - 1 mm or 10 µm -0.5 mm.

In other examples, at least one of the conductive anode 106, the conductive cathode 108, or the reference node may be printed on the substrate using conductive printing techniques. For example, at least one of the conductive anode 106, the conductive cathode 108, or the reference node 112 may be printed on the substrate 104 using screen-printing techniques, using a (conductive) ink-jet printer, and the like. It is to be appreciated that any other deposition or incorporation methods may be used to form the conductive anode 106, conductive cathode 108, and the reference node 112 on or within the substrate 104.

Generally, the conductive anode 106, the conductive cathode 108 and the reference node 112 are comprised of biocompatible electrically-conductive materials. Examples of such materials include silver, silver chloride, silver compounds, gold, gold compounds, platinum, platinum compounds, and/or binary alloys of platinum, nickel, cobalt or palladium with phosphorus, or binary alloys of platinum, nickel, cobalt or palladium with boron, and the like. Non-metallic materials are also contemplated for electrode formation such as conductive polymers and the like. Conductive polymers can include, but are not limited to, polyaniline, polythiophene, polypyrrole, polyphenylene, poly(phenylenevinylene), and the like.

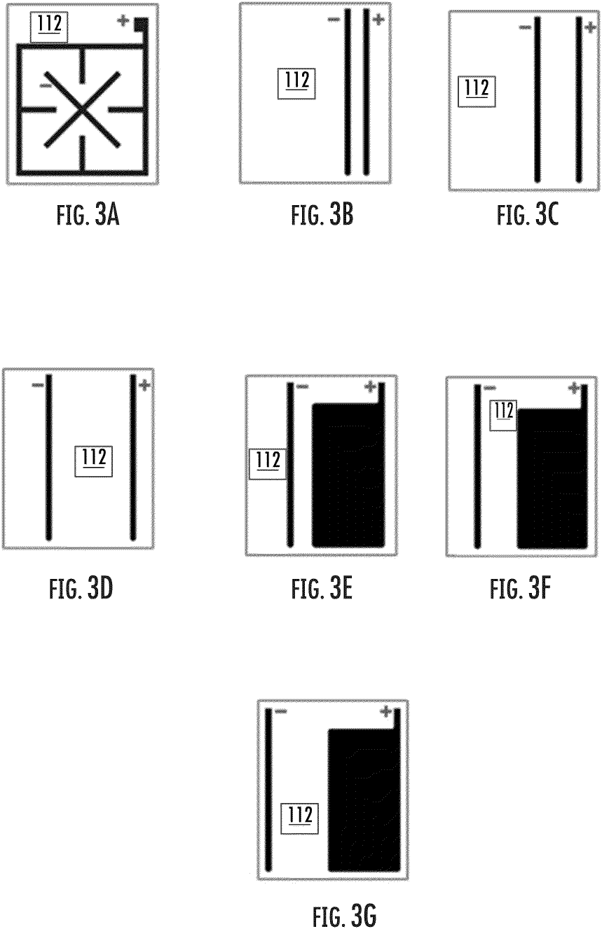

The conductive anode 106, the conductive cathode 108, and the reference node 112 may be of any size and/or shape. Generally; however, as shown in FIG. 2B the conductive anode 106 is larger than the conductive cathode 108. In one example, the conductive anode 106 substantially covers the wound. For example, the conductive anode 106 can be conformed to a shape such that it substantially covers the wound. Similarly, the size and shape of at least one of the conductive anode 106 and the conductive cathode 108 can be determined by at least one of wound size, wound shape, and location of the wound. Likewise, the size and shape of the electroceutical dressing 100 can be determined by at least one of wound size, wound shape, and location of the wound. The electroceutical dressings can be formed into any of a number of possible shapes, patterns or geometries, depending upon the application and topography of the wound or application site. Any aspect of the wound dressing can be manufactured in a variety of shapes and configurations. For example, configurations can include, but are not limited to, compressive wraps, tampons, tubular, roll gauze, pads of varying sizes and shapes, island dressings, strip dressings, dressings for dental applications, rectal dressings, vaginal pads, surgical packing or dressings, or any combination thereof. Spacing between the conductive anode 106 and the conductive cathode 108 may also depend upon wound size, location and shape. Generally, it is desired to have the conductive anode 106 and conductive cathode 108 as close together as possible without causing an electrical short-circuit. Exemplary feature spacings include, without limitation, from about 10 nm to about 10 mm, from about 10 nm to about 200 µm, from about 100 nm to about 100 µm, from about 1 µm to about 100 µm, or from about 1 µm to about 100 µm. FIGS. 3A-3G are non-limiting examples of various size, spacing and shapes of the conductive anodes 106 (shown with the “+” signs in FIGS. 3A-3G), conductive cathodes (shown with the “-” signs in FIGS. 3A-3G), and reference nodes 112, although the electrodes can take any of a variety of appropriate configurations. The disclosed electroceutical dressings can include a configuration of electrodes 106, 108 that can generate both shallow (e.g., 1 µm - 2 mm, or 1 µm - 1 mm), and deep (e.g., 1 mm - 100 mm, or 2 mm -100 mm) currents into a wound, or a combination thereof.

Referring to FIGS. 2A and 2B, the energy source 110 causes a potential difference between the anode 106 and the cathode 108. In various embodiments, the energy source 110 may be switched or otherwise controlled where control includes at least its on/off state, its voltage, and/or the current that is allowed to flow from the conductive anode 106, through the wound 102, to the conductive cathode 108. As shown in FIG. 2A, at least a portion of the anode 106 is in contact with at least a portion of the wound 102 or exudate 202 of the wound. Similarly, at least a portion of the cathode 108 and/or the reference node 112 is in contact with at least a portion of the wound 102 or exudate 202 of the wound. It is desirable for the wound 102 or the exudate 202 to be moist. Moisture of the wound 102 or the exudate 202 reduces electrical resistance and facilitates flow of the electrical current through the wound 102. The moisture may be from the natural secretions and exudate that is inherent to a wound, or it may be from moisture that is added to the wound. For example, the wound 102 may be moistened with sterile saline, water, gels, antibiotic creams and lotions, and the like. Such moistening agents may be applied directly to the wound 102 before placement of the electroceutical dressing 100. In other aspects, the electroceutical dressing 100 may include means for moistening the wound 102. For example, as shown in FIG. 2C, a reservoir 204 with appropriate tubing 206 may be used to apply moisture to the wound 102 while the electroceutical dressing 100 is in place. The tubing may be configured such that the rate of moisture flowing from the reservoir 204 to the wound 102 can be controlled to avoid saturating the substrate 104. Though shown located on the substrate 104, it is to be appreciated that the reservoir 204 may be located separate from the substrate 104. Any other means may also be used for moistening the wound 102. As electrochemical reactions occur in the wound bed and at the electrodes (anode 106 and cathode 108), the total system electrical resistance (dressing + wound bed) changes over time and the potential at each electrode 106, 108 changes and therefore there is no direct control over the amount and rate of HOC1 production. To address this challenge, reference electrode 112 is provided so that the working electrode (i.e., the anode) 108 is at a fixed potential with respect to the reference electrode 112, thus HOC1 can be generated through well-known, controlled electrochemical reactions.

As shown in FIGS. 1, and 2A-2D, the electroceutical dressing 100 includes an energy source 110. The energy source 110 may be attached to the substrate 104 (see FIG. 2B), or all or a portion of the energy source may be separate from the substrate 104 (see FIG. 2D). The energy source 110 can comprise any device or any means for creating a potential difference between the conductive anode 106 and the conductive cathode 108. Generally, the potential difference is at approximately 6 volts, though voltages of other values may also be used. The energy source 110 may be an alternating-current (AC) or a direct current (DC) source, or combinations thereof. The energy source 110 may be an AC source rectified to become DC, or a DC source inverted to become AC. Frequency dependent energy source 110 can have any of a variety of appropriate wave forms including, but not limited to, square, sinusoidal, triangular, trapezoidal, or more complex patterns. AC sources can be modulated in any of a variety of ways including temporally and spatially.

In one aspect, the energy source 110 comprises a battery, which may include any number of cells connected in either series and/or parallel. In one aspect, the energy source 110 may comprise a battery paired with an inverter to create an AC source or it may be an AC source transformed to the desired voltage. In one exemplary embodiment, the energy source 110 connected to the at least one electrode pair comprises an energy harvesting device that creates the voltage differential between the anode 106 and the cathode 108. For example, the energy harvesting device may be of the type that creates the voltage differential between the anode 106 and the cathode 108 based on movement of a subject to whom the electroceutical dressing is applied. Further, the energy source 110 may at least in part use a galvanic reaction between the anode 106, the wound 102, and the cathode 108 to create a voltage differential between the anode 106 and the cathode 108, which results in the electrical current flowing from the conductive anode 106, through the wound 102, to the conductive cathode 108.

FIG. 2E illustrates another embodiment of an electroceutical dressing 100 having an alternate form of an energy source 110. In this example, the energy source 110 generally connected to the anode 106 and cathode 108 comprises a plurality of electrodes 208 in a spaced pattern on the substrate that are substantially in contact with the wound 102 and/or the exudate 202. A first subset 210 of the plurality of electrodes 208 are connected in electrical series and/or electrically parallel with the anode 106 and a second subset 212 of the plurality of electrodes 208 are connected in electrical series and/or electrically parallel with the cathode 108. Generally, the plurality of electrodes 208 are comprised of a biocompatible reduction/oxidation reaction material such that a voltage differential is created between the anode 106 and the cathode 108, wherein the voltage differential causes the electrical current to flow from the conductive anode 106, through the wound 102, to the conductive cathode 108. The number, spacing and materials used in the electrodes 208 can vary and at least these parameters can be used to determine the voltage differential between the anode 106 and the cathode 108. For example, the biocompatible reduction/oxidation reaction materials that comprise the electrodes 208 may be silver and zinc such that the first subset 208 of the plurality of electrodes 210 are comprised of silver and the second subset 212 of the plurality of electrodes 208 are comprised of zinc, or vice-versa. Other biocompatible reduction/oxidation reaction materials may be used to form the electrodes 208. Generally, the voltage potential created between each spaced electrode 208 pair is about 0.2 volts, but as noted herein this can vary depending upon several parameters including, spacing, materials, contact with the wound 102, moisture of the wound 102, and the like.

FIG. 2F illustrates an embodiment of an electroceutical dressing such as the one shown in FIG. 2E, that further comprises a plurality of unconnected electrodes 214 in a spaced pattern on the substrate 104 that are electrically isolated from one another on the substrate 104 and are substantially in contact with the wound 102 and/or the exudate 202. The plurality of unconnected electrodes 214 are comprised of a biocompatible reduction/oxidation reaction material such that an electrical field is created in and around the wound 102 by the plurality of unconnected electrodes 214, thus facilitating wound healing. It is to be appreciated that although FIG. 2F shows the energy source 110 comprising a plurality of electrodes 208 in a spaced pattern on the substrate 104, the plurality of unconnected electrodes 214 in a spaced pattern on the substrate 104 that are electrically isolated from one another on the substrate 104 are not limited to this embodiment. The unconnected electrodes 214 can be used with any energy source 110 that create a potential difference between the anode 106 and the cathode 108 including a battery, an AC source, a DC source, an energy harvesting device, and the like.

In one aspect, as shown in FIG. 2G, the electroceutical dressing 100 can optionally include a barrier 216 that covers a part of or substantially covers a side of the substrate 104 opposite the wound 104. Among other things, the barrier 216 is configured to maintain a desired moisture level of the wound 102 and/or prevent contamination of the substrate 104 and the electroceutical dressing 100. Generally, the barrier 216 is waterproof or water-resistant such that moisture is retained within the wound 102 and outside moisture is prevented from entering the wound 102. The barrier 216 can be any biocompatible semi-permeable or impermeable material for limiting the evaporation of moisture from the substrate 104 and/or the wound surface. The barrier 216 can be fixedly attached to the substrate 104 and/or the skin of the subject or removably attached for easy removal and replacement.

The barrier 216 can control the rate of moisture evaporation from the substrate and/or the wound 102, and also function as a physical barrier to the penetration of microbes from the surrounding environment. The barrier 216 can be a film, fabric or foam. Some preferred materials include, but are not limited to, polyurethanes, polyolefins such as linear low density polyethylene, low density polyethylene, ethylene vinyl acetate, vinylidene, chloride copolymer of vinyl chloride, methyl acrylate or methyl methacrylate copolymers and combinations thereof. A preferred polymeric material is polyurethane, either as a film or as a polyurethane foam. The polyurethane may be an ester or ether based polyurethane. Materials suitable for a foam moisture regulation layer can be any semi-permeable or impermeable natural or synthetic compound including, but not limited to, rubber, silicon, polyurethane, polyethylene polyvinyl, polyolefin, hydrogels, or combinations thereof.

Alternatively, the barrier 216 may be a transparent elastomer film for visual inspection of the moisture status of the substrate 104. The film can have a thickness from 10 µm to 100 µm. The barrier 216 may have an MVTR of from about 300 to about 5,000 grams/meter2/24 hours, preferably from about 800 to about 2,000 grams/meter2/24 hours. The barrier 216 can be laminated to the substrate 104 by methods well recognized in the art.

The electroceutical dressing 100 can optionally include one or more therapeutic agents. Exemplary therapeutic agents include, but are not limited to, growth factors, analgesics (e.g., an NSAID, a COX-2 inhibitor, an opioid, a glucocorticoid agent, a steroid, or a mineralocorticoid agent), antibiotics, antifungals, anti-inflammatory agents, antimicrobials (e.g., chlorhexidine-, iodine-, or silver- based agents), antiseptics (e.g., an alcohol, a quaternary ammonium compound), antiproliferative agents, emollients, hemostatic agents, procoagulative agents, anticoagulative agents, immune modulators, proteins, vitamins, and the like.

FIG. 2H illustrates an example of an electroceutical dressing 100 that further comprise a current-limiting element 220, as known in the art. In one aspect, the current-limiting element may be integrated with an into the energy source 110. In other aspects, it is a stand-alone element. The current-limiting element 220 may be, for example, an inductor, resistors or a resistor bridge, a current-limiting circuit, and the like. The purpose of the current limiting element is to limit the electrical current flowing from the conductive anode 106, through the wound 102, to the conductive cathode 108 to a desired range or to less than a maximum value. For example, the desired range of the current flowing through the wound 102 may be 15 milliamps, or less. In other aspects, the desired range of the current flowing through the wound 102 may be 10 milliamps, or less. Generally, current is limited so that an unacceptable level of heating caused by the electrical current flowing from the conductive anode 106, through the wound 102, to the conductive cathode 108 is not experienced. For example, it may be desired that power density applied to the wound is at or below governmentally-regulated standards to prevent unacceptable heating in the wound. In the United States, such regulations may be promulgated by the Food and Drug Administration (FDA). For example, power density may be required to be at or below approximately 0.25 W/cm² to avoid unacceptable levels of heating. Though not shown in FIG. 2H, other embodiments of the electroceutical dressing 100 may include, for example, a voltage indicator that indicates voltage is being provided by the energy source 110 connected to the at least one electrode pair 106, 108 and/or a current indicator that indicates the presence of electrical current flowing from the conductive anode 106, through the wound 102, to the conductive cathode 108.

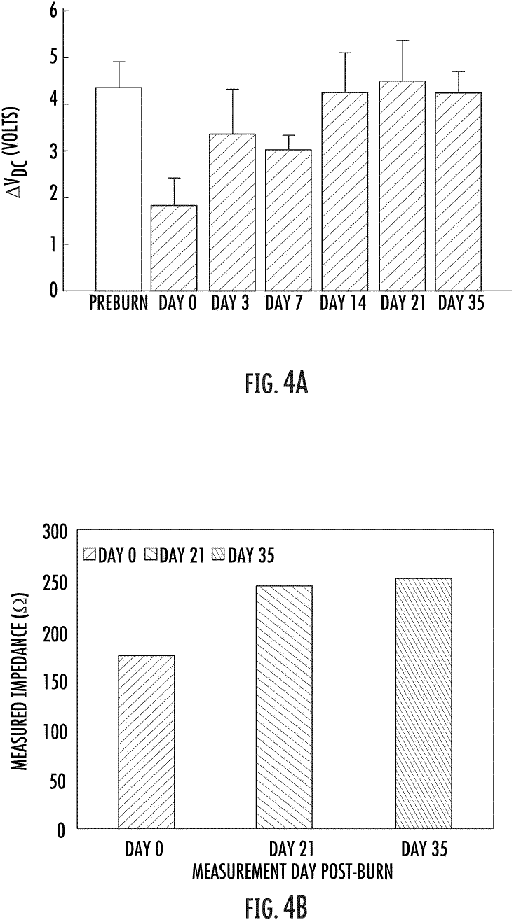

Optionally, other embodiments of the electroceutical dressing 100 may include one or more sensors. To control electroceutical actuation for HOC1 generation, it is desired to know at what stage of healing the wound is, at a given point in time (with respect to the healing stages). The current standard method that exists to monitor wound healing is a visual inspection of the wound by the clinician. This method is very less deterministic and varies from person to person. Hence, a quantifiable parameter is desired that can be monitored in real time and does not require removal of the dressings frequently for inspection. To monitor both the biochemical and physical states across distinct regimes of angiogenesis, inflammation control, and wound closure, one or more sensors can be integrated into embodiments of the electroceutical dressing, including: (i) miniaturized existing TEWL (transepidermal water loss) probes (measures temperature and humidity to estimate wound evaporation rate); (ii) electrical impedance and/or eddy current sensors to provide a detailed ‘geographical map’ of the wound bed electrical properties in real time. TEWL measurements are a reliable, existing method to verify the restoration skin barrier function and also serve as a measure of validation of the remaining sensors that may be integrated into an embodiment of the electroceutical dressing. The impedance and eddy current sensors rely on data that shows that as infected, burn wounds healed with application of an embodiment of the disclosed electroceutical dressing, the electrical properties of the wound tissue evolved in time (see FIGS. 4A, and 4B). As shown, the electrical impedance increased as the wound healing progressed, suggesting that electrical properties of the wound bed can be used to assess the level of wound healing and closure.

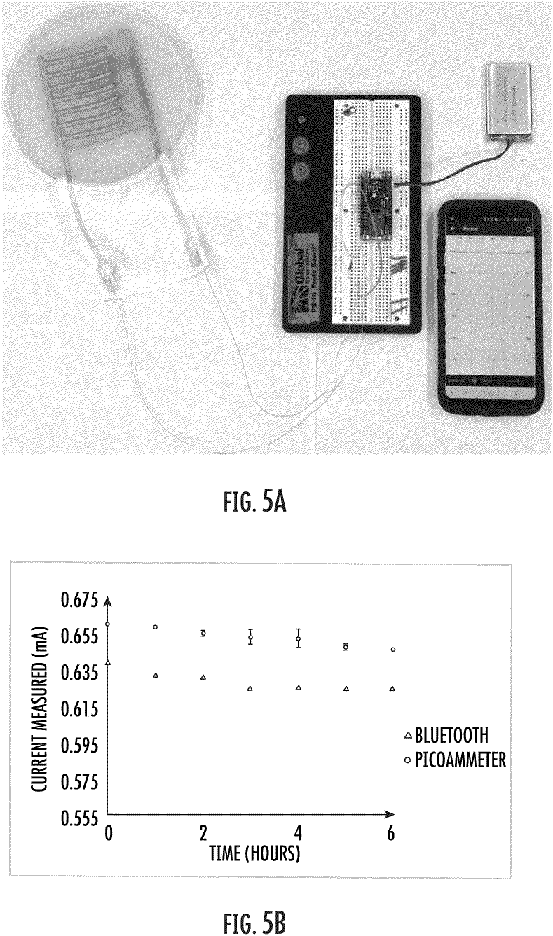

In addition, to avoid the frequent removal of the dressings, the sensors may use wireless communications (see FIGS. 5A and 5B). FIG. 5A is a photograph of an embodiment of the electroceutical dressing interfaced with Bluetooth transmission circuit and the electronic device (in this case mobile) receiving the data from the Bluetooth. FIG. 5B illustrates current data recorded by the Bluetooth and compared against the current data recorded by a picoammeter. Other form of wireless communication may be used in addition to Bluetooth.

n one aspect, the one or more sensors may comprise an electrical impedance spectroscopy (EIS) probe that senses impedance through the wound 102. In another aspect, the sensor may comprise an eddy current probe. As noted herein, the measured impedance through the wound 102 can be used to detect wound healing. For example, as shown in FIG. 2I, EIS measurements on exemplary pig burn wounds were shown to increase and level off as the wound healed. Non-contact eddy current measurements were also performed on exemplary pig burn wounds. Eddy current measurement is performed by first recording voltage in air away from wound (null measurement) for 5 seconds. The eddy current probe is then brought into contact with the wound and the voltage is recorded for another 5 seconds. The results of these measurements are shown in FIG. 2J. A single measurement is recorded as |ΔVDC| which is the absolute difference between the average of the two measurements. Each bar is an average of 5 measurements on 6 different wound sites (Total 30 measurements). Data are mean + SD; p<0.01 vs pre-burn; p<0.05 vs day 0.

FIG. 2K is an illustration that shows biofilm 222, as described herein, formed in the wound 102. Biofilm 222 can be resistant to antibiotics and other forms of medicinal treatment. As shown in FIG. 2J, the electrical current flowing from the conductive anode 106, through the wound 102, to the conductive cathode 108 can inhibit biofilm 222 formation within the wound 102. Furthermore, the electrical current flowing from the conductive anode 106, through the wound 102, to the conductive cathode 108 can at least partially disrupt a biofilm 222 growing within the wound. Even bacteria that has not formed a biofilm can be disrupted and/or destroyed by the electroceutical dressing. The electrical current flowing from the conductive anode 106, through the wound 102, to the conductive cathode 108 can at least partially destroys sessile and/or planktonic bacteria within the wound 102.

The electroceutical dressings disclosed herein can be fabricated by various methods. The electrodes can be fabricated from the conductive materials disclosed herein.

In an exemplary embodiment, the electroceutical dressing fabrication process can begin with applying the conductive electrodes 106, 108, 112 to the substrate 104. This step may involve printing techniques such as screen-printing or using an ink-jet printer, among other methods. For example, applying the electrodes may comprise screen printing Ag/AgCl ink (cathode) and a paste of zinc (Zn) powder and poly (vinyl alcohol) (PVA) in water (anode) on a silk fabric.

Also, in certain embodiments, electrodes 106, 108, 112 can be fabricated by stamping a solution of conductive polymer or precursor(s) thereof onto the substrate 104. Any of a variety of known methods for stamping can be used to fabricate the electrodes. In certain embodiments, electrodes 106, 108, 112 can be fabricated using a capillary micromolding technique and/or apparatus. In certain embodiments, electrodes, 108, 112 can be fabricated by printing conductive polymer and/or prepolymer directly onto an appropriate substrate 104. In one example an ordinary laser printer is used in combination with specially formulated ink to form a patterned conductive polymer film. An appropriate ink formulation can comprise a conductive polymer and/or prepolymer thereof. Additionally, such an ink may optionally comprise a binder, a surfactant, and/or an oxidizing agent such as ferric ethylbenzenesulfonate. In one example, a substrate 104 coated with an appropriate ink is exposed to excess monomer vapor thereby developing the image in the regions containing oxidizing agent. This results in a conductive polymer image. In certain embodiments, a laser printer can be used to print a negative image of an electrode. The negative can then be dipped into a conductive polymer deposition/coating system. This results in polymer coating both the negative image and the exposed substrate 104. Then the image can be developed by removing the toner. In one example, a negative image of an interdigitated electrode (IDE) can be printed on an ordinary overhead transparency using a laser printer. The conductive polymer can then be formed in situ. In certain embodiments, electrodes can be fabricated photolithographically.

The layers of disclosed electroceutical dressings may or may not be attached to each other or can be provided as a component of another structure. For example, an electrode including a patterned conductive layer on a base substrate can be applied directly to the affected site, such as a wound. The energy source can be integral with or supplied separately from the electrode assembly of the electroceutical dressings.

The disclosed electroceutical dressings can be used to treat wounds of an animal or human subject. The appropriate aspect of the wound dressing can be selected and positioned on the wound, with the electrodes in direct contact or indirect contact with the wound.

In one aspect, disclosed is a method of treating or preventing a bacterial infection (e.g., a biofilm infection) in a wound, the method including applying a therapeutically effective amount of an electric current to the wound. The electric current can be applied to the wound via a electroceutical dressing as disclosed herein. The electroceutical dressing can include an anode and a cathode that are substantially in contact with the wound or its exudate such that an electric current flows through the wound. As a non-limiting example, the voltage potential between the anode and the cathode may be from 1-10 volts. Potential between the anode 106 and the reference node 112 can be monitored to ensure optimal HOCL generation in the wound. The time of treatment may range from hours to days. The electroceutical dressing can be applied, for example, within 4-6 hours of injury to prevent biofilm formation. The electroceutical dressing can be applied, for example, after biofilm formation (e.g., 7 days after injury) to treat a biofilm infection. The method may reduce the bacterial load by > 90% over a period of 4 weeks. For example, the starting bacterial load may be 10⁵-10⁸ colony forming units (cfu)/ml, where 10⁵ is the clinical infection threshold, and the method of treatment using the electroceutical dressing reduces the bacterial load to below the clinical threshold (e.g. at or below 10¹-10² cfu/ml) in the wound.

FIG. 6 is an illustration of an exemplary three-electrode electroceutical dressing. In this embodiment, a layer of solid material (i.e., any material that is substantially an insulator in its solid state and is substantially a conductor in its moistened or dissolved state and forms a conductive solution) is sandwiched between the substrate of the electroceutical dressing and a substrate of an energy source (in this instance, a flexible fabric battery having reduction/oxidation reaction material electrodes, e.g., Zn/Ag battery). Generally, the solid insulating material is placed on the second side of the substrate of the electroceutical dressing or within the substrate of the electroceutical dressing such that the solid insulating material is between the at least one of the conductive anode and the conductive cathode and the energy source. The layer of solid material (e.g., NaCl, KCl, sugar, glucose, and the like) serves an electrolyte when it is moistened (e.g., comes in contact with the wound fluid, wound exudate, blood from the wound, externally applied water, saline, alcohol (including PVA), and the like). The flexible fabric battery operates once the wound fluid (or external water, saline spray, etc.) seeps through the substrate of the dressing and starts dissolving the solid layer, thus producing a conducting liquid. In turn, this creates a conducive, moist environment that triggers the electrochemical reactions needed to generate electrical potential and power the dressing and integrated sensors that comprise the electroceutical dressing.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications may be referenced. The disclosures of these publications in their entireties are hereby incorporated by reference into this application in order to more fully describe the state of the art to which the methods and systems pertain and to illustrate improvements over the present state of the art in claimed invention.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

1. An electroceutical dressing comprising:
a substrate having two sides, wherein one of the sides is a wound-facing side and a second side opposite the wound-facing side;
three or more electrodes positioned on the wound-facing side of the substrate, said electrodes comprising at least a conductive anode, a conductive cathode, and a conductive reference node, wherein each of three or more electrodes are configured to be in at least partial contact with a wound when the electroceutical dressing is placed on the wound; and
an energy source having a positive terminal and a negative terminal, wherein the positive terminal is configured to connect to one of the conductive anode and/or the conductive cathode, and the negative terminal is configured to connect to an other of the conductive cathode and/or the conductive anode when the electroceutical dressing is placed on the wound, wherein when connected to the conductive anode and the conductive cathode the energy source induces an electrical current to flow through the wound.

2. (canceled)

3. (canceled)

4. (canceled)

5. (canceled)

6. (canceled)

7. (canceled)

8. (canceled)

9. (canceled)

10. The electroceutical dressing of claim 1, further comprising one or more sensors comprised of one or more of a transepidermal water loss probe, an electrical impedance sensor, and an eddy current sensor, wherein a change in sensed electrical characteristics of the electrical impedance sensor and the eddy current sensor are used to estimate healing of the wound.

11. (canceled)

12. (canceled)

13. (canceled)

14. (canceled)

15. The electroceutical dressing of claim 1, wherein a change in electrical characteristics of the energy source and/or the three or more electrodes over time is used to estimate healing, wherein a change in electrical characteristics of the energy source and/or the three or more electrodes includes a change in resistance and/or impedance between any two of the electrodes, and/or a change in current and/or voltage of the energy source.

16. (canceled)

17. (canceled)

18. (canceled)

19. (canceled)

20. (canceled)

21. The electroceutical dressing of claim 1, further comprising an insulating material, wherein the energy source is substantially insulated from at least one of the conductive anode and the conductive cathode using the insulating material, wherein the insulating material is substantially an electrical insulator when in its solid form but when moistened forms an electrically-conductive solution, wherein the insulating material comprises NaCl, KCl, sugar, glucose, or any other material that presents electrically isolating properties in the solid phase and upon exposure to a solvent dissolves entirely or in-part to generate an electrically conducting solution.

22. (canceled)

23. (canceled)

24. (canceled)

25. (canceled)

26. (canceled)

27. (canceled)

28. (canceled)

29. (canceled)

30. The electroceutical dressing of claim 1, wherein the conductive anode is larger than the conductive cathode.

31. (canceled)

32. The electroceutical dressing of claim 30, wherein the conductive anode is conformed to a shape such that it substantially covers the wound.

33. The electroceutical dressing of claim 1, wherein a size and shape of the electroceutical dressing is determined by at least one of wound size, wound shape, and location of the wound.

34. The electroceutical dressing of claim 1, wherein a size and shape of at least one of the conductive anode and the conductive cathode is determined by at least one of wound size, wound shape, and location of the wound.

35. (canceled)

36. (canceled)

37. (canceled)

38. (canceled)

39. (canceled)

40. (canceled)

41. (canceled)

42. (canceled)

43. The electroceutical dressing of claim 1, wherein the energy source comprises at least two electrodes in a spaced pattern on an insulating substrate and the at least two electrodes are comprised of a biocompatible reduction/oxidation reaction materials such that a voltage differential is created between the at least two electrodes.

44. The electroceutical dressing of claim 43, wherein the energy source comprises a plurality of electrodes in a spaced pattern on the insulating substrate wherein a first subset of the plurality of electrodes are connected in electrical series and a second set of the plurality of electrodes are connected in a separate electrical series and the plurality of electrodes are comprised of a biocompatible reduction/oxidation reaction material such that a voltage differential is created between the first subset of the plurality of electrodes connected in electrical series and the second subset of the plurality of electrodes connected in electrical series.

45. (canceled)

46. (canceled)

47. (canceled)

48. (canceled)

49. (canceled)

50. (canceled)

51. (canceled)

52. The electroceutical dressing of claim 1, further comprising a barrier that substantially covers the second side of the substrate, said barrier configured to maintain a desired moisture level of the wound.

53. (canceled)

54. The electroceutical dressing of claim 1, further comprising a current-limiting element, wherein the current limiting element limits the electrical current flowing through the wound to a desired range.

55. (canceled)

56. (canceled)

57. (canceled)

58. (canceled)

59. The electroceutical dressing of claim 1, further comprising a voltage indicator, wherein the voltage indicator indicates voltage being provided by the energy source, and/or a current indicator, wherein the current indicator indicates a presence and/or a magnitude of the electrical current flowing through the wound.

60. (canceled)

61. The electroceutical dressing of claim 1, wherein at least one of the three or more electrodes are comprised of a biocompatible reduction/oxidation reaction material.

62. The electroceutical dressing of claim 61, wherein the electrical current flowing through the wound inhibits formation of a biofilm within the wound, at least partially disrupts a biofilm growing within the wound, at least partially destroys sessile bacteria within the wound, and/or at least partially destroys planktonic bacteria within the wound.

63. (canceled)

64. (canceled)

65. (canceled)

66. (canceled)

67. (canceled)

68. (canceled)

69. (canceled)

70. (canceled)

71. A method of assembling an electroceutical dressing, comprising:
applying a layer of conductive material to an insulated base substrate to form at least one electrode pair and a reference electrode on the insulated base substrate, wherein the pair of electrodes comprise a conductive anode and a conductive cathode that are electrically insulated from one another and the reference electrode is insulated from the conductive anode and the conductive cathode,
wherein an energy source is connected to the at least one electrode pair, wherein the energy source induces an electrical current to flow from the conductive anode, through a wound, to the conductive cathode,
wherein potential between the anode and the reference electrode is monitored and/or controlled to produce HOCL in the wound.

72. (canceled)

73. (canceled)

74. (canceled)

75. (canceled)

76. (canceled)

77. (canceled)

78. (canceled)

79. (canceled)

80. The method of claim 71, wherein the energy source connected to the at least one electrode pair comprises a flexible fabric battery and the flexible fabric battery comprises a plurality of electrodes in a spaced pattern on a fabric substrate, wherein the energy source comprises the plurality of electrodes in a spaced pattern on the fabric substrate and a first subset of the plurality of electrodes are connected in electrical series and a second set of the plurality of electrodes are connected in electrical series and the plurality of electrodes are comprised of a biocompatible reduction/oxidation reaction material such that a voltage differential is created between the anode and the cathode.

81. (canceled)

82. (canceled)

83. (canceled)

84. (canceled)

85. (canceled)

86. The method of claim 71, further comprising an insulating material, wherein the energy source is substantially insulated from at least one of the conductive anode and the conductive cathode using the insulating material, wherein the insulating material comprises NaCl, KCl, sugar, glucose, or any other material that presents electrically isolating properties in the solid phase and upon exposure to a solvent dissolves entirely or in-part to generate an electrically conducting solution.

87. (canceled)

88. The method of claim 71, further comprising providing a current-limiting element, wherein the current limiting element limits the electrical current flowing from the conductive anode, through the wound, to the conductive cathode to a desired range.

89. (canceled)

90. (canceled)

91. (canceled)

92. The method of claim 71, further comprising providing one or more sensors, wherein the one or more sensors measure impedance through the wound or eddy current in the wound, which is used to determine healing of the wound.

93. (canceled)
